(19) United States
(12) Patent Application Publication
(10) Pub. No.: US 2013/0172807 A1
(43) Pub. Date: Jul. 4, 2013

(54) Peritoneal Dialysis Catheter

(71) Applicant: Cosme Cruz, Chicago, IL (US)

(72) Inventor: Cosme Cruz, Chicago, IL (US)

(21) Appl. No.: 13/478,732

(22) Filed: May 23, 2012

Related U.S. Application Data
(60) Provisional application No. 61/490,287, filed on May 26, 2011.

(51) Int. Cl. A61M 1/28

(52) U.S. Cl. CPC A61M 1/285
USPC 604/29

(57) ABSTRACT

A refined catheter for percutaneous access to the peritoneal cavity for the deliver/removal of fluid in medical procedure such as peritoneal dialysis including a flexible tubular member having a distal portion that features either a coiled segment with multiple secondary ports and/or a bolus tip with a large scalloped orifice on the side, and a proximal segment that can be oriented cephalad or caudally by means of a 90° angle and a connecting portion with a luer fitting and an inner diameter of at least 0.375 cm is maintained (3.75 mm) through the entire length of the catheter to optimize its flow rate capabilities.

### FIELD OF THE INVENTION

The invention relates generally to catheters. More specifically, the invention relates to catheters useful in peritoneal dialysis procedures.

### BACKGROUND OF THE INVENTION

In the treatment of various diseases, percutaneous access to the peritoneal cavity is necessary. One example is peritoneal dialysis which is often indicated for acute or chronic renal failure. To effect dialysis through the peritoneal cavity, a percutaneous passage is surgically formed through the cutaneous and subcutaneous tissues, rectus muscle and through the peritoneum itself. This passage permits insertion and implantation of a distal portion of the catheter within the peritoneal cavity. A separate tunnel directed either caudally or cephallad is then formed through the subcutaneous and cutaneous tissues with a tunnel exit site in the suprapubic region of the external abdominal wall, or the presternal area if so preferred. A proximal portion of the catheter is inserted through this tunnel, thereby maintaining an end of the proximal portion in a downward direction along the abdominal wall. Examples of catheters used in peritoneal dialysis are disclosed in U.S. Pats. Nos. 3,633,585; 4,184,497; 4,278,092; 4,279,252; 4,392,855; 4,687,471, 4,772,269; and 4,935,004.

Over the last two decades there has been a greater understanding of the function of the peritoneal dialysis catheter and its interaction with the dialysis systems used in terms of its hydraulic function; ease of operation and maintenance. Modern catheters, Twardowski's (U.S. Pat. No. 4,772,269) swan neck and Cruz's (U.S. Pat. No. 4,935,004) pail handle, signify major advances in catheters. For example, these devices address the issue of catheter failure caused by outflow obstruction either from dislocation within the peritoneal space or occlusion caused by omental tissue or other intra-abdominal organs.

The materials used for their manufacture, their design and specifications give each catheter its character, and usually drives the operability of the device including implanting and function once in place.

As new, more efficient dialysis delivery systems become available, the need for similarly efficient catheters becomes more apparent. Additionally the patients' input in selecting among various dialysis technologies is increasingly encouraged. As the consumer learns of options in terms of dialysis modality (for example, hemodialysis versus peritoneal dialysis and in center versus home dialysis), the need to offer devices that will suit them in terms of function and comfort becomes prominent. Efficiency, unemcumberance from garments and aesthetic qualities of the catheter have been recently recognized as important by patients and caregivers.

As can be seen, design, materials, implantation into the human body and maintaining hygiene and sterility while the catheter is in the environment of use all continue to present ongoing issues. With an ongoing emphasis on reducing costs and returning patients to a home treatment environment, these concerns persist. Even with patient training there is a need for procedures and devices which enable proper patient care and self care.

Once the patient returns to the home-environment, the healthcare professional loses sight and control over the general environment of treatment. While economies of scale are forcing healthcare, especially dialysis, into less expensive treatment environments, there are countervailing considerations. For example, the age and mental acuity, among other factors, of the patient is a consideration in the training of the patient to properly execute their own care. Further, the complexity of the care required needs to be measured in moving the patient home from the clinic or hospital environment. Other factors include the maintenance or monitoring of devices implanted in the patient as well as instruments that have been placed in the home for the patient's use in treatment.

If the proper care is not taken in execution of the treatment, the goal of economizing in care is lost with the need for further medical treatment. Profilactically training, providing supplies and periodic monitoring of patient, investments in the treatment environment all assist in accomplishing stated goals.

Additionally, the design and implementation of devices to be inserted into the patient are also of concern. Ensuring the integrity of in vivo devices—which by their very nature are reused—is of paramount concern. Many considerations come into play as discussed above including the proper placement and sealing of these devices within the body cavity. Additionally, the positioning of these devices from distal to proximal portions so that they are comfortable and secure with continuing use and with the activities of daily life.

Another paramount concern in the design and implementation of these devices is sterility not only during dialysis but also when not in use. For example, dialysis patients may suffer incontinence either due to age or medical condition. Diapers can provide an environment which fosters bacteria is less than acceptable to devices such as catheters. Further disabilities such as ostomies or other physical impairment may make certain types of bathing a preference if not a requirement.

Hence, while previous catheters have solved certain concerns, there is a need for further developments which foster advances in the care of dialysis patients.

### SUMMARY OF THE INVENTION

Prior to the development of the present invention, a need existed for a catheter for percutaneous access to the peritoneal cavity in which distal and proximal portions of the catheter are ultimately caudally directed. The proximal segment of the catheter is to be used with the 180° bend that would dictate the caudal orientation of the exiting segment. The orientation of the site where the catheter exits the skin affects the long-term functional life of the catheter. The area or environment where the skin and catheter interface must be structurally sound and stable. Thus with patients in the erect posture the catheter would not suffer the stress of gravity or tugging. Further, a need existed for a peritoneal dialysis catheter which would securely anchor within the rectus muscle tissue without use of anchoring beads or flanges. A need also existed for a peritoneal dialysis catheter in which the distal and proximal portions of the catheter were non-planar to thereby function as a safeguard from inadvertent dislodging of the catheter from the patient. This invention describes a catheter with flow capabilities such as to efficiently move fluid at a minimum rate of one liter per minute without causing jet stream related discomfort. The invention provides a catheter which exits above the waistline which at the same time provides an optional hood to cover the proximal end of the catheter to ensure sterility.

Benefits of an exit site located in the thorax include the ability for patients to bathe in a tub without the catheter exit site soaking in stagnant water, its use in patients with urinary/fecal incontinence wearing diapers, patients with colostomy/ileostomy infants and anyone needing to wear a diaper.

Other advantages and aspects of the invention will become apparent upon making reference to the specification, claims, and drawings to follow.

### BRIEF DESCRIPTION OF THE DRAWINGS

FIG. 1 is a front elevational view of a dialysis catheter.

FIG. 2 is a partial frontal view of the catheter shown in FIG. 1.

FIG. 3 is a partial side elevational view of the catheter shown in FIGS. 1 and 2.

FIG. 4 is a frontal elevational view of a catheter shown in accordance with a preferred embodiment of the invention.

FIG. 5 is a partial frontal view of the catheter of FIG. 4.

FIG. 6 is a partial side elevational view of the cathether shown in FIG. 4.

FIG. 7 is a partial side elevational view of a bolus tip in accordance with one aspect of the invention.

FIG. 8 is a partial top elevational view of the bolus tip shown in FIG. 7.

FIGS. 9A and 9B are alternative embodiments of the invention showing a partial cutaway side view of the proximal end of the catheter covered by a protective hood (FIG. 9A) and a cutaway top plan view of the catheter of the proximal end of the invention covered by a protective hood (FIG. 9B).

FIG. 10. is a view depicting the catheter in its environments of use.

### DETAILED DESCRIPTION OF THE INVENTION

This invention is susceptible of illustration in varying embodiments and in many different forms. There is shown in the drawings and will herein be described in detail a preferred embodiment of the invention. The present disclosure is to be considered as an exemplification of the principles of the invention and is not intended to limit the broad aspect of the invention to the embodiment illustrated.

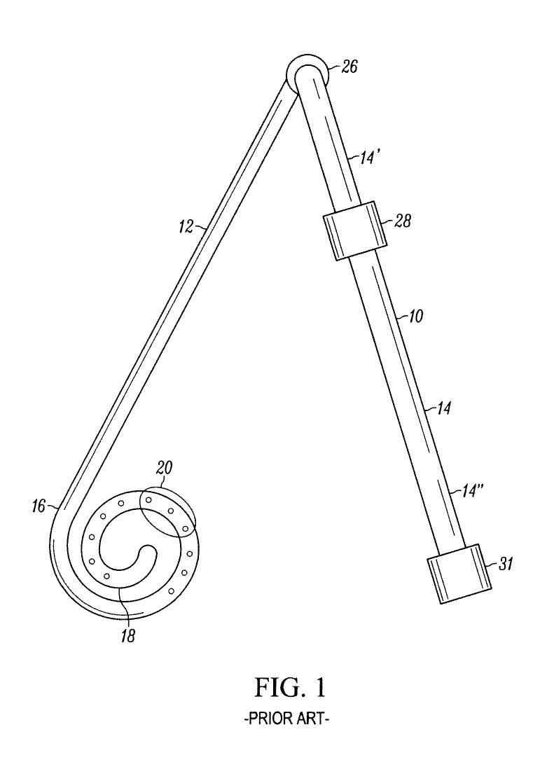

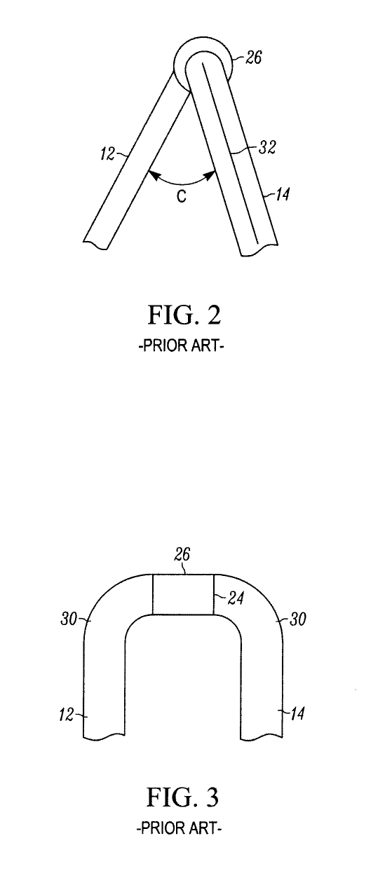

Referring now to the drawings, FIGS. 1-3 disclose one embodiment of a prior catheter 10 comprised of flexible tubing, preferably a thermoplastic material such as polyurethane. Thermoplastic material is preferred over silicone because such material may re-form at body temperatures enabling the catheter to conform in vivo to internal body contours. Thus avoiding device related stress on the surrounding tissues by device “memory”. Thermoset materials, such as silicone, cannot be so modified. The polyurethanes also by virtue of their greater strength allow thinner walls and larger ID than silicone catheters with similar external diameters.

The catheter 10 is comprised of a distal portion 12 which, as later explained, will extend into the peritoneal cavity of the patient and a proximal portion 14. As will be later explained, a first segment 14′ of proximal portion 14 passes through the subcutaneous and cutaneous tissue of a patient, whereas a second segment 14″ extends externally from the patient and downwardly along the abdominal wall exiting through skin at a site below the belt line.

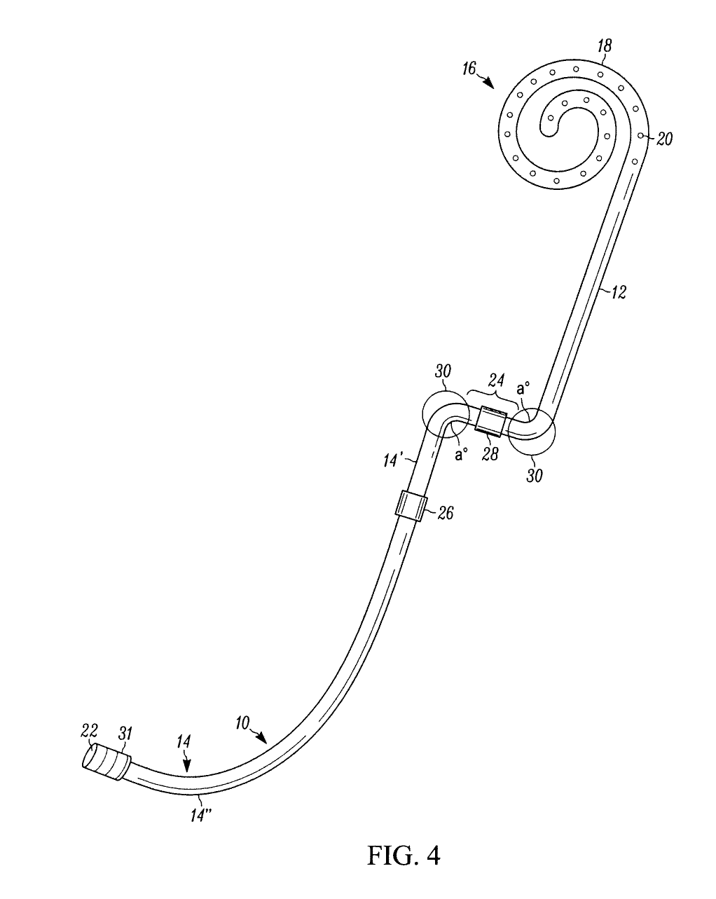

In accordance with the invention, FIGS. 4-6 depict a catheter which is designed to provide an ascending portion along the anterior wall of the trunk to exit in the pre-sternal region after undergoing a 180° turn. As with the catheter of FIGS. 1-3, the catheter of FIGS. 4-6 may be directed to define a caudal exit of the device through the tegument.

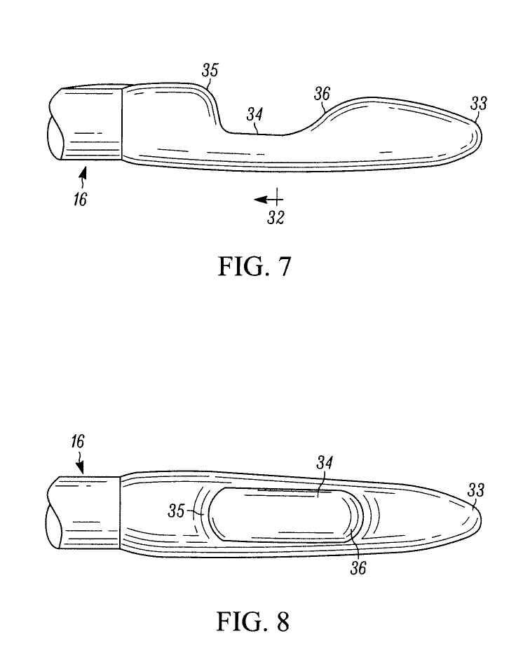

Distal portion 12 includes a distal end 16 which, in a preferred embodiment, comprises a spiral or coiled configuration 18 which lies in a single plane when the catheter is in a natural, unstressed condition. Spiral 18 includes a plurality of openings 20 which permits passage of fluid such as a dialysate. The invention may add about one inch to this portion 18 and the “bolus” tip, FIGS. 7 and 8, to enhance the flow capability by decreasing the resistance to flow; flow equaling pressure over resistance.

The need for high flow capable devices with minimum “whipping” or jet stream forces are desirable the catheter of the invention may have an optional “bolus” tip with a side line opening in a shape to decrease the force of the fluid column as it exits and makes it flow around the tip. This bolus makes the installation and withdrawal of large volumes of fluid (dialysate) without pain/discomfort possible. As seen in FIG. 7 the outer diameter of the bolus 32 decreases from the area where it joins the catheter body 14 to the nose section 33. The bolus body 32 generally 3 times the external diameter of catheter 14. Body 14 and bolus 32 could be formed as one piece through an insert injection molding process such that the bolus 32 is integrally formed with the catheter 14.

The passage 34 forms a single fluid ingress and egress for the bolus 32 and the catheter 14. As best seen in FIGS. 7 and 8 the shape of port 34 is formed by removing a piece of the body 32 around greater than 180° of the circumference of the body 32. The port 34 extends substantially around 190° of the circumference of the body 32. As a result fluid delivered is dispensed and the discomfort caused by jet forces abates or disappears directly opposite to the center of the port 34. The flow of the fluid passage is built up progressively 35 and 36, in the nose section 33 of the bolus body 32 so as to define a uniform arc terminating at the outer surface of the body 33. Other features of the bolus are known from U.S. Pat. No. 5,571,093 which is incorporated herein by reference.

Proximal portion 14 has a proximal end 31 which may be coupled to a source of dialysate and/or a container for dialysate drainage. In all embodiments of the present invention, distal portion 12 and proximal portion 14 are preferably linear in configuration. Distal portion 12 and proximal portion 14 are linked by a linear connecting portion 24. Connecting portion 24 has a length which is substantially co-extensive with the thickness of the rectus muscle. As a result, connecting portion 24 is substantially embedded within the rectus muscle upon implantation of catheter 10. In all embodiments of the present invention, connecting portion 24 carries about its circumference a porous cuff 26 which preferably extends for about 1 cm of the length of connecting portion 14. Cuff 26 may be made from a woven relatively bioincompatible material known in the art, such as Dacron® Velour. Cuff 26 results in a thorough embedding and anchoring of connecting portion 14 within the rectus muscle. An optional additional porous cuff 28 may also be carried on proximal portion 24.

Distal portion 12 and proximal portion 14 are intrically formed with and angularly joined to connecting portion 24. Preferably, the angular joinder of distal portion 12 and proximal portion 14 to connecting portion 24 is achieved by molding bend segments 30 into catheter 10. As a result of such molding process, bend segments 30 remain in catheter 10 even when catheter 10 is in its natural unstressed condition. Proximate to each bend 30 is an angle a°, FIG. 4. As molded, angle a° is approximately 90°. Generally cuff 28 is equidistant between the bends 30 on span 24. Angle a° formed between the longitudinal axis of the connecting portion and longitudinal axis of distal portion 12 should be such that when catheter 10 is implanted into a patient, distal portion 12 is directed caudally into the peritoneal cavity. Likewise, angle a° should be such that upon implantation of catheter 10 into a patient, proximal end 22 is directed cephalade (upwardly) along the external abdominal wall of the patient. To assure such caudal direction of distal portion 12 and upward direction of proximal portion 14, such angles (a°) are preferably 90° angles.

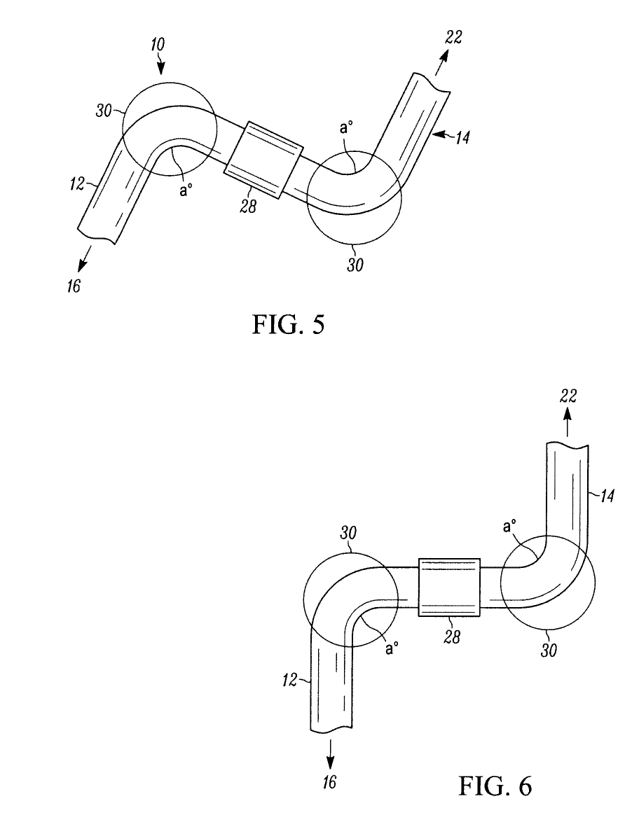

Preferably, distal portion 12 and proximal portion 14 are non-planar with each other, FIGS. 5 and 6. Unlike prior art peritoneal catheters in which distal and proximal catheter portions are co-planar, the present invention uses non-planar distal and proximal portions to prevent inadvertent withdrawing or dislodging the catheter from the patient and relieve stress of “tugging”. Specifically, if the proximal portion 14 of the catheter 10 is pulled upon, the non-planar and angular orientation of catheter 10, FIGS. 4-6, will prevent withdrawal of the catheter from the patient by abutting against the inner surfaces of the peritoneal cavity.

Catheter 10 may include a longitudinal, radio-opaque stripe as is known in the art, for ease of implantation and for x-ray confirmation of catheter position during implantation. This stripe (not shown) in the unstressed configuration will be consistently oriented to the 12 o'clock position.

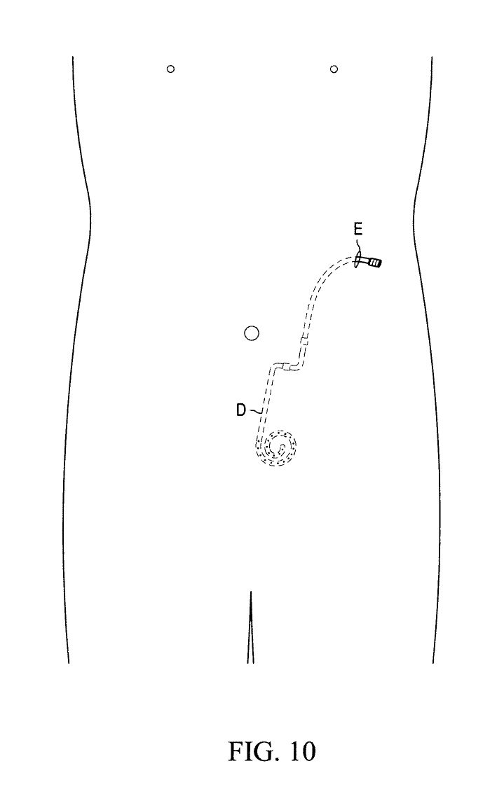

In order to minimize tunnel infection and tunnel exit site infection, it is essential that distal end 16 of distal portion 12 be directed caudally within the peritoneal cavity and that proximal end 22 of proximal portion 14 also be downwardly directed along the external abdomen wall, FIG. 10. One advantage of the invention is that the catheter emerges from the body in the upper abdominal region well above the waistline. This allows use of the catheter with PD patients that have any number of complications such as incontinence problems the need for tub bathing, as well as any other environmental or use concerns which might otherwise lead to contamination of the catheter.

Catheter 10 may be implanted by any technique known in the art. For example, in the “Y-Tec” method, distal portion 12 and connecting portion 24 are inserted through a surgically formed percutaneous passage into the peritoneal cavity. Next, a tunnel is formed through the subcutaneous and cutaneous tissue which exits through a second opening formed in the epidermis. The proximal portion 14 may then be urged through the tunnel so that first segment 14′ passes through the subcutaneous and cutaneous tissue while second segment 14″, FIG. 4, is positioned external to the abdomen wall. The tunnel assures that proximal portion 14 is maintained in a caudal direction. Finally, the percutaneous passage to the peritoneal cavity is sutured closed.

The non-planar orientation of distal portion 12 and proximal portion 14, are angularly off-set. Hence, if external force is exerted on proximal portion 14, catheter 10 will not easily be dislodged or withdrawn from the patient. In prior art catheters having co-planar distal and proximal portions, such force is imparted directly to the distal portion 12 causing dislodgement of the catheter. Distal portion 12, by being non-planar with proximal portion 14, will merely abut against the inner surfaces of the peritoneal cavity to prevent withdrawal or dislodgement of catheter 10.

FIG. 5 discloses the significance of the configuration of connecting portion 14. As disclosed in FIG. 5, the length of connecting portion 14 is longer and is generally co-extensive with the thickness of the patients rectus muscle. Preferably, porous cuff 28 is also co-extensive with the length of connecting portion 24. Hence, connecting portion 24 is imbedded in rectus muscle tissue which, as previously mentioned, efficaciously grows into the pores of cuff 28. Bent segment 30 on distal portion 12 caudally directs distal end 16 into the peritoneal cavity FIG. 10. Likewise, bent segment 30 on proximal portion 14, with the assistance of a surgically formed tunnel, downwardly directs first segment 14′ through subcutaneous tissue which emerges through the epidermis. Second segment 14′ emerges caudally at an exit site E. A second porous cuff 26 may be carried on first segment 14″ to permit ingrowth of subcutaneous tissue. However, in some instances, the embedding and efficacious anchoring of cuff 28 into rectus muscle may prove sufficient. As positioned once implanted, the proximal portion of the catheter exits the abdominal wall cephalad presternal off of the midline, FIG. 10.

Additionally the invention incorporates the presence of an integral adaptor 22 which maintains an interior diameter of 3.75 mm and enhances flow capabilities by eliminating the flow restricting retracing effects of “mounted on” adaptors. These “built in” connecting segments prevent accidental disconnection. In another embodiment of the present invention the distal end of the distal portion may be linear, rather than comprising the spiral configuration disclosed in FIG. 4.

The device of the invention has an inner diameter of 3.75 mm. Having at least 3.75 mm inner diameter throughout, provides a tremendous impact in flow capability, (Bernoulli's principle). For example, considering improving dialysis efficiency, such as in the case of Cyler assisted dialysis where on the average ten short cycles are done automatically but currently used systems with poor flow spend too much of the time moving fluid in and out of the patient's abdomen. For example, time savings for most patients doing manual exchanges may present a differential where instead of spending 40 minutes or more on dialysis, dialysis exchanges may be completed in less than seven minutes.

Preliminary clinical investigation has revealed no subsequent tunnel site or tunnel exit site infection many months after catheter implantation which indicates that upon ingrowth of rectus muscle tissue into porous cuff 26 on connecting portion 24, catheter migration is eliminated. Finally, despite the absence of any anchoring buttons or flanges such as those found in the prior art, initial observations have revealed no catheters which have become inadvertently dislodged or withdrawn.

The goal of achieving a “stable” device/catheter/tegument/interface is accomplished with the invention which brings us closer to the idea a stable cutaneo/peritoneal fistula for medical treatment purposes.

This invention is classified as a “simple” device according to Cruz classification (Cruz, C., et al. “Access for Peritoneal Dialysis in Clinical Nephrology”, Dialysis & Transplants”, Mallucche, H., Sawaya, B. P., Hakim, Rm et al. Eds. Pp. 1-18, Delsemhosen II, 1999. Some of the New Catheters are Better, Seminars in Dialysis V, 3. 202-4 1992)) simple catheter can be implanted by minimally invasive methods (percutaneously or openly). Percutaneous methods include laparoscopy or “blind” methods. Regardless of the method of implantation, the key step is the positioning of the proximal cuff 26 in the midst of the rectus abdominal muscle. A “purse string” suture of heavy “O” absorbable material will secure this position without fear of dislodgement until the in-growth of tissue gives structural support to the catheter.

The radio-opaque reference line, positioned at 12 o'clock, ensures the distal part 12 is and remains in the pelvis. Before the proximal 14 segment is exteriorized a dialysis exchange is performed to verify the hydraulic function of the catheter. If the function is deemed acceptable, the exit site and tunnel are created in conventional manner such as by using a tunneling device which will accompany the catheter packaging.

Following the catheter is capped and irrigated on day one and seven after insertion. Ideally continued use of the catheter ought to be delayed for 2 weeks. The Catheter Adapter 31 will have an external luer compatible with all the dialysis administration sets available. This “universal” connector will obviate the need to stock more than one “system” and keep the inner diameter of the entire system consistently at 3.75 mm resulting in improved flow rates.

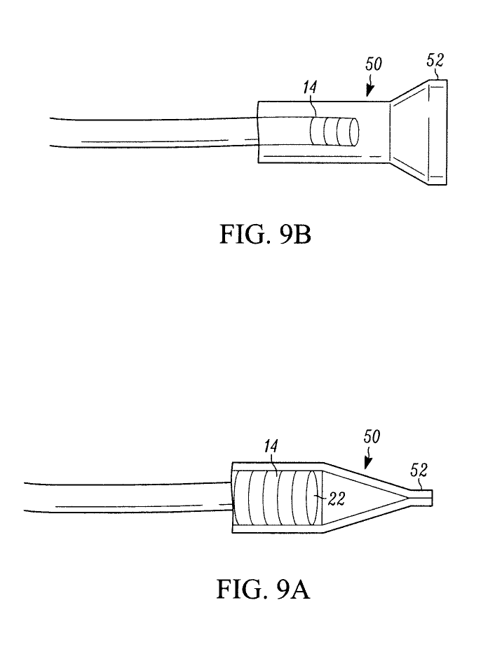

In accordance with a further embodiment of the invention, there is shown in FIGS. 9A and 9B. As can be seen the proximal end 14 of the catheter 10 may be covered by the hood 50. The hood 50 may be molded from any number of materials to provide a sealing edge 52. The sealing edge 52 may be molded to close through any number of means such as grooved flanges or a closure. Sealing edge 52 functions to protect the sterility of proximal portion 14 and more specifically the integral adaptor 22. When in use the hood may be pulled back to expose the adaptor 22.

While the specific embodiments have been illustrated and described, numerous modifications come to mind without significantly departing from the spirit of the invention and the scope of protection is only limited by the scope of the accompanying

The claimed invention is:

1. A catheter being adapted for percutaneous access to a peritoneal cavity of a patient, such as in peritoneal dialysis, comprising:
a flexible tubular member having a distal portion adapted to be disposed within the peritoneal cavity, the distal portion having an open distal end;
a proximal portion of the tubular member having one segment being adapted for passing through the subcutaneous and cutaneous tissues of the patient and an other segment adapted to be disposed along the external abdominal wall of the patient and terminating in a proximal end presternal off the midline;
a linear connecting portion between the distal portion and the proximal portion, the connecting portion adapted to be substantially disposed through the rectus muscle of the patient, the connecting portion being adapted to have a length generally co-extensive with the thickness of the rectus muscle;
porous cuff means being carried on the connecting portion; and,
the catheter having a preformed and unstressed configuration with the distal portion being angularly joined to the connecting portion to direct the distal end caudally within the peritoneal cavity, the proximal portion being angularly joined to the connecting portion to direct the proximal end upwardly along the external abdominal wall, the distal portion and the proximal portion being non-planar with each other.
